(19) United States
(12) Patent Application Publication
(10) Pub. No.: US 2003/0152918 A1
(43) Pub. Date: Aug. 14, 2003

(54) BRCAI AND HMLHI GENE PRIMER SEQUENCES AND METHOD FOR TESTING

(76) Inventor: JAN VIJG, SAN ANTONIO, TX (US)

Correspondence Address: ROBERT H RINES, RINES AND RINES, ATTORNEYS AT LAW, 81 NORTH STATE STREET, CONCORD, NH, 03301

(21) Appl. No.: 09/306,333

(22) Filed: May 6, 1999

Related U.S. Application Data
(60) Provisional application No. 60/084,408, filed on May 6, 1998.

(51) Int. Cl. C12Q 1/68; C07H 21/04; C12P 19/34

(52) U.S. Cl. 435/6; 435/91.2; 536/24.3

(57) ABSTRACT

Primer sequences and materials are pre-prepared as test kits for enabling appropriate gene scanning patterns, preferably by two dimensional electrophoresis (TDGS), for use in detecting sequence variations and/or mutations in BRCA1 and h MLH1 genes

The present application is based upon provisional application No. 60/084408, filed May 6, 1998, and is directed to methods of and primer sequences for sequence variation and/or mutation detection of BRCA and hMLH1 genes, such as by two-dimensional denaturing gradient electrophoresis techniques (TDGS).

### BACKGROUND

Such techniques are described in Method Of And Apparatus For Diagnostic DNA Testing, Jan Vijg and Daizong Li, PCT/IB96/00543, filed Jun. 3, 1996, International Publication Number WO96/39535, Dec. 12, 1996, and in “Two-Dimensional DNA Typing”, Molecular Bio Technology, Vol. 4, 1995, pp 275-295

The tests leading to the establishment of the primer sequences for the BRCA1 and hMLH1 of the present invention were conducted with the TDGS design prepared with the computer programming and equipment described in PCT/IB97/00976, published on or about Feb. 14, 1998.

### OBJECTS OF INVENTION AND SUMMARY

The objects of the invention are to provide novel theoretically and empirically (experimentally) derived TDGS patterns for hMLH1 and BRCA1 genes which may be used by testers to test for gene sequence variation and/or mutations.

### DRAWINGS

FIGS. 1A and 1B show the computer-aided design TDGS patterns obtained for the hMLH1 and BRCA1 (theoretical-left hand side, empirical or experimental—right hand side)

In the theoretical vs. empirical patterns of the MLHI and BRCA1 genes, for all four genes, one or more exons were designed in overlapping fragments, in which case the fragment name is exon. 1, exon..2, etc. Exons 8 and 15 of hMLH1 contain polymorphisms, which can be distinguished from disease-causing heterozygous mutations on the basis of a unique four-spot pattern (18).

### DESCRIPTION OF THE INVENTION IN PREFERRED FORMS

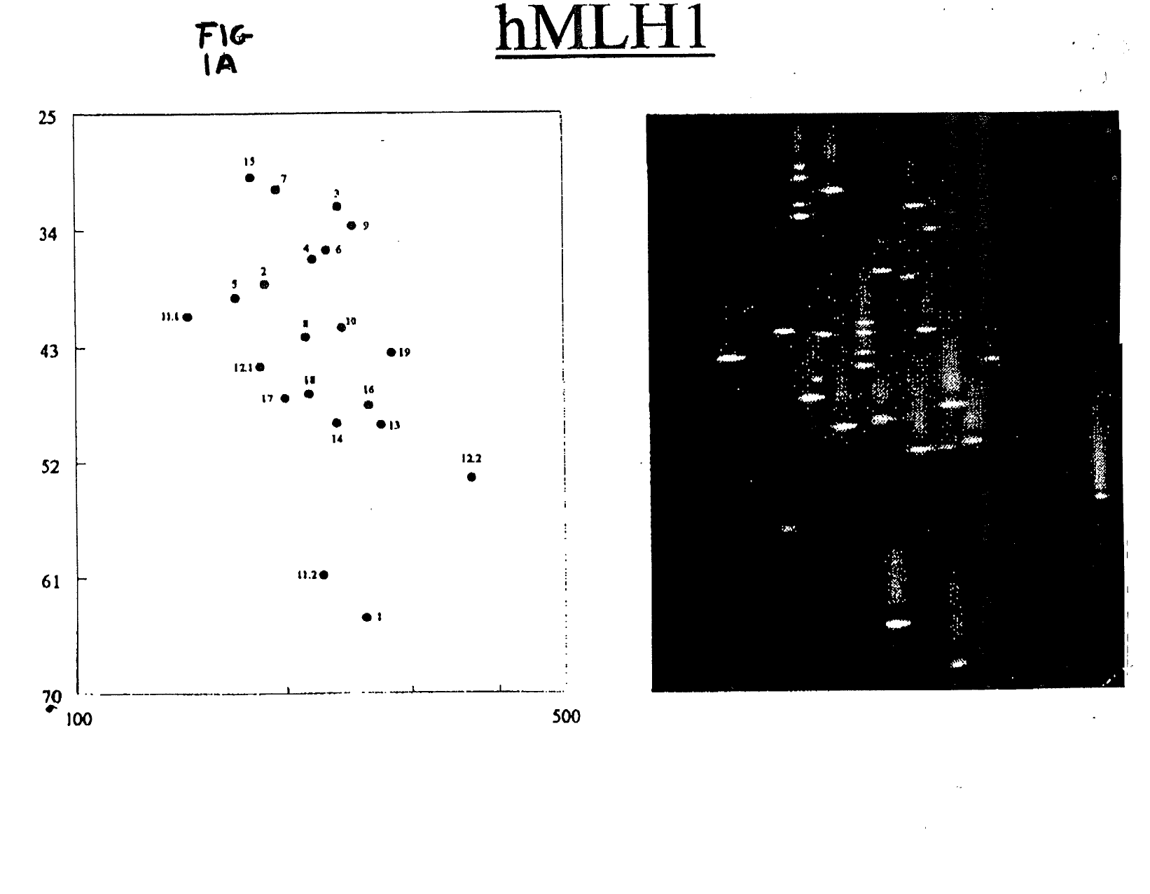

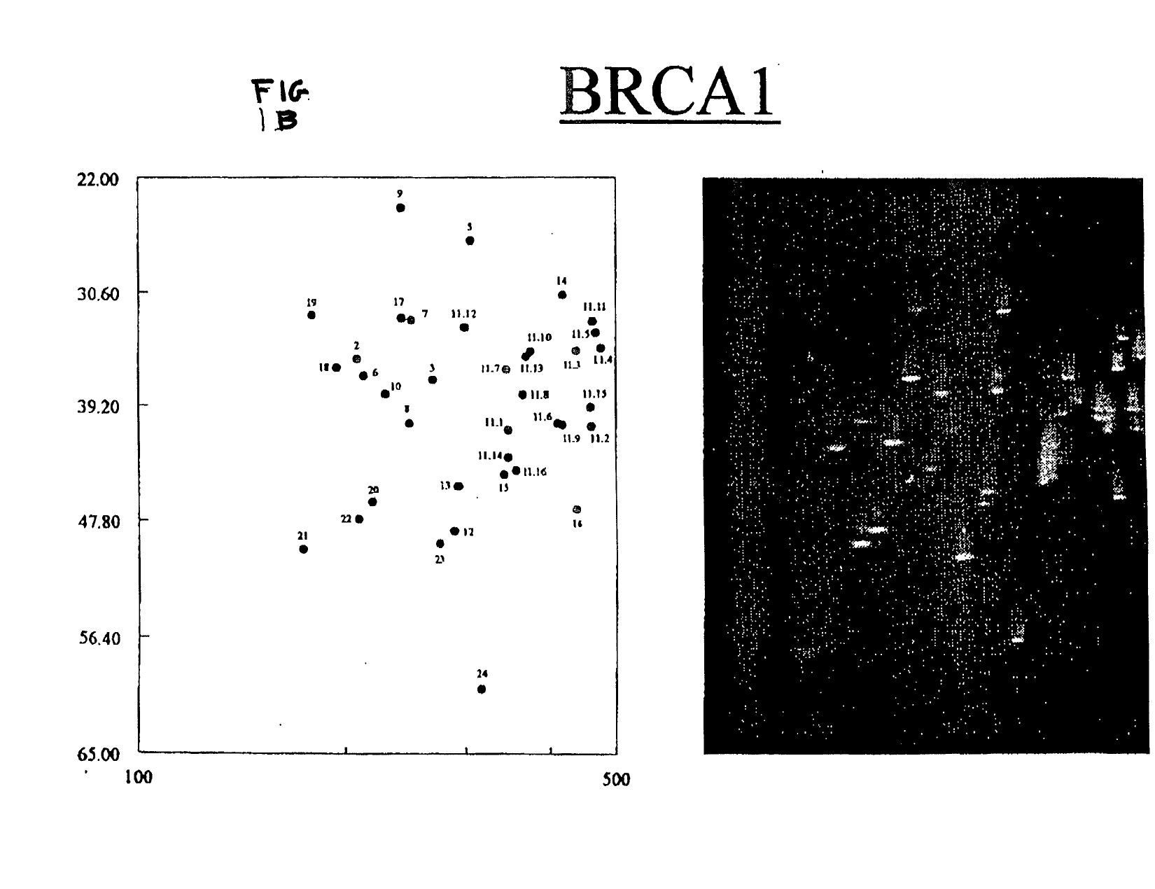

The MLH1 DNA mismatch repair gene, The design for MLHI took 30 minutes (excluding exon indication). FIG. 1A shows the theoretical and the empirical TDGS patter for the MLHI gene. Because exons 11 and 12 had to be subdivided into overlapping fragments, two multiplex groups are currently being used, with the long PCR carried out as a four-plex PCR. Like many other genes, exon 1 of MLH1 is GC-rich and, hence, was found to melt at a much higher % UF compared to most of the other fragments. Thus far, a total of 41 coded samples with previously identified mutations have been analyzed in a blinded fashion with 100% concordance (30).

The breast and ovarian cancer susceptibility gene BRCA1. The tumor suppressor gene BRCA1 contains 24 exons, of which exon 11 contains approximately 60% of the coding region. FIG. 1B shows the theoretical and empirical 2-D pattern for BRCA1. Of all 2-D designs discussed, this was the most difficult (total design time was 2 h), the main reason being the need to make overlapping fragments for the 3.4 kb exon 11. Pre-amplification was accomplished by one 7-plex long PCR. Using the long PCR amplicons as template, all 24 exons were amplified in a total of 37 fragments distributed over 5 multiplex groups. The overlap and sometimes short distances from fragment to fragment necessitated the use of so many multiplex short PCR groups. The non-coding exons 1a, 1b and the non-coding part of exon 24 were excluded. Evaluation of this test design using a panel of coded samples with previously identified mutations is currently ongoing. Thus far, mutations and polymorphisms have been detected in exons 2, 8, 11, 16, 20 and 23.

PCR Amplification

Primers were obtained from Genosys Biotechnologies, Inc. (The Woodlands, Tex.). For complete lists of all sequences, except BRCA1, see references 18, 29 and 30. Primer sequences for BRCA1 will be published elsewhere but will be made available upon request. PCR amplification of gene sequences was carried out using the two-step protocol first described by Li and Vijg (22). Primers for long-distance PCR were designed based on published sequences (24-27) using Primer Designer 3, to amplify the entire gene-coding region for each of the 4 genes as a 1-plex (TPS3), a 6-plex PCR (RBI), a 4-plex PCR (MLHI) or a 7-plex PCR (BRCA1). The LA PCR kit (Takara) was used for long PCR in a PTC-100 thermocycler (M J Research). Multiplex short PCR was carried out using the long PCR products as template. Between 0.1 and 1.125 μM of each primer was used in a 50 μl reaction with 1 μl of long PCR product in 20 mM Tris-HCl (pH 8.4), 50 mM KCl, 250 μM of each dNTP and 5% formamide. Two and a half units of Taq DNA polymerase (Life Technologies) were added after an initial denaturation at 94° C. for 60 s. Cycling conditions for multiples short PCR and concentrations of MgCl₂ varied among different, genes and amplifications were carried out in a PTC-100 thermocycler (M J Research).

Two-Dimensional DNA Electrophoresis

For RBI, 5 μl of multiplex short PCR was used per electrophoresis run. For TP53, MLHI and BRCA1. 5 μl of each of the different multiplex groups were combine. One tent of a volume of loading buffer (0.25% xylene evanol. 0.25% bromophenol blue, 15% ficoll and 100 mM Na₂EDTA) was added and the mixtures were loaded onto a 6.5% (TP53) or 10% (RB1, MLHI and BRCA1) PAA non-denaturing size gel (acrylamide: bisacrylamide =3755:1) in 0.5×TAE buffer. The samples were electrophoresed for 5.3 h at 150 V (RBI), 5 h at 120 V (TP53) or 7.5 h at 140 V (MLHI and BRCA1) at 50° C. After staining the gel into a mixture of equal amounts of SYBR-green I and II (Molecular Probes, Eugene, Oreg.) for 20 min, the region containing all fragments of interest (usually between 100 and 600 bp) was cut out and loaded onto a denaturing gradient get (DGGE). Gradients used were 0 to 50% UF for RBI, 20 to 70% UF for TP53, 25 to 70% UF for MLH1 and 20 to 65% UF for BRCA1. The second orthogonal dimension was run for 12 h at 100V (RBI), 14 h at 120 V (TP53) or 16 h at 100 V (MLHI and BRCA1). Spot patterns were visualized by SYBR-green staining using a FluorImager (Molecular Dynamics, Sunnyvale, Calif.).

6 The primer sequences for long and short PCR for the BRCA1 are as follows:

A. Primer Pairs for Long Distance PCR

Exons 1-4
MLH1-4F
GCG.GCT.AAG.CTA.CAG.CTG.AAG.GAA.GAA.CGT.GA
MLH1-4R
GGC.GAG.ACA.GGA.TTA.CTC.TGA.GAC.CTA.GGC.CC
product size = 10.8 kb
Exons 5-10
MLH5-10F
GCG.CCC.CTT.GGG.ATT.AGT.ATC.TAT.CTC.TCT.ACT.GG
MLH5-10R
GCG.CTC.ATC.TCT.TTC.AAA.GAG.GAG.AGC.CTG
product size = 10.5 kb
Exons 11-13
MLH11-13F
CGG.CTT.TTT.CTC.CCC.CTC.CCA.CTA.TCT.AAG.G
MLH11-13R
GGG.TTA.GTA.AAG.GAA.GAG.GAG.CTT.GCC.C
product size = 8.7 kb
Exons 14-19
MLH14-19F
GGT.GCT.TTG.GTC.AAT.GAA.GTG.GGG.TTG.GTA.G
MLH14-19R
GCG.CGC.GTA.TGT.TGG.TAC.ACT.TTG.TAT.ATC.ACA.C
product size = 10.5 kb

Underlined nucleotides represent nucleotides added
to modify melting temperatures of the primers

B. Primer Pairs for Short PCR

| | | Pro- | | |
|---|---|---|---|---|
| Exan | | duct | | |
| Clamp ¹ | Size | Tm² | Primer Sequence | |
| 12.1 | 40 | 184 | 44.53 | TTT.TTT.TTT.TTT.TAA.TAC.A |
| | | | | AAT.CTG.TAC.GAA.CCA.TCT |
| 12.2 | 8 | 366 | 53.23 | TGG.AAG.TAG.TGA.TAA.GGT |
| | 40 | | | TGT.ACT.TTT.CCC.AAA.AGG |
| 13 | 40 | 272 | 49.06 | ATC.TGC.ACT.TCC.TTT.TCT |
| | | | | AAA.ACC.TTG.GCA.GTT.GAG |
| 14 | 45 | 235 | 48.94 | TAC.TTA.CCT.GTT.TTT.TGG |
| | 5 | | | GTA.GTA.GCT.CTG.CTT.GTT |
| 15 | 40 | 179 | 29.97 | CAG.CTT.TTC.CTT.AAA.GTC |
| | | | | CAG.TTG.AAA.TGT.CAG.AAG |
| 16 | | 261 | 47.56 | CTT.GCT.CCT.TCA.TGT.TCT.TG |
| | 40 | | | AGA.AGT.ATA.AGA.ATG.GCT.GTC |
| 17 | 40 | 199 | 47.01 | ATT.ATT.TCT.TGT.TCC.CTT |
| | | | | AAT.GCT.TAG.TAT.CTG.CCT |
| 18 | 45 | 215 | 46.67 | CCT.ATT.TTG.AGG.TAT.TGA.AT |
| | | | | GCC.AGT.GTG.CAT.CAC.CA |
| 19.1 | | 282 | 43.43 | TGT.TGG.GAT.GCA.AAC.AGG |
| | 40 | | | ATC.CCA.CAG.TGC.ATA.AAT |

¹GC clamps:
50 clamp: CGC.CCG.CCG.CCG.CCC.GCC.GCG.CCC.CGC.GCC.
CGT.CCC.GCC.GCC.CCC.GCC.CG
45 GC clamp: CGC.CCG.CCG.CGC.CCC.GCG.CCC.GTC.CCG.CCG.CCC.CCG.CCC.GGC.CCG
40 clamp: CGC.CCG.CCG.CGC.CCC.GCG.CCC.GGC.CCG.CCG.CCC.CCG.CCC.G
8 clamp: CGT.CCC.GC
5 clamp: GCG.CG
2 clamp: CG
²Tm is given in % UF

9 The primer sequences for long and short PCR for the BRCA1 are as follows:

| Primers for long-PCR BRCA1 | |
|---|---|
| (7-PLEX) | |
| BR1/1-3F: | TgT ACC TTg ATT TCg TAT TCT gAg Agg CTg CTg CTT Ag |
| BR1/1-3R: | gAg AAA gAA TgA AAT ggA gTT ggA TTT TCg TTC TCA C |
| Size: | 9.9 kb |
| BR1/5-9F: | TAg CCA TgA AAA gAT AAT CTC ACA ACT gCC CTT AAg AgC |
| BR1/5-9R: | ACC AgC CTA CTT gAg ggA ggA Agg Tgg gAA gA |
| Size: | 9.7 kb |
| BR1/10-11F: | gAg AgC AgC TTT CAC TAA CTA AAT AAg ATT ggT CAg CTT TCT gT |
| BR1/10-11R: | TCA AgT TTA AgA AgC AgT TCC TTT AAC TAT ACT Tgg AAA TTT gT |
| Size: | 4.8 kb |
| BR1/12-13F: | gCT Agg ACg TCA TCT TTg gCT TgA ATg AgC TTT A |
| BR1/12-13R: | gCg ATA ATT ACC CAT gTg CTg AgC AAg gAT CA |
| Size: | 9.0 kb |
| BR1/14-17F: | TCT TCA ATg Tgg Agg CAg TAg ggA Tgg AgA AA |
| BR1/14-17R: | ggg TCT CCA ggT TTT gCC TCA CTT gTT CTT TC |
| Size: | 10.7 kb |
| BR1/18-20F: | TCT TAA CTT CAT ATC AgC CTC CCC TAg ACT TCC AAA TAT CC |
| BR1/18-20R: | CAT CTC TgC AAA ggg gAg Tgg AAT ACA gAg Tg |
| Size: | 7.2 kb |
| BR1/21-24F: | CAC TCT TCC ATC CCA ACC ACA TAA ATA AgT ATT gTC TC |
| BR1/21-24R: | gCA TAg CCA gAA gTC CTT TTC Agg CTg ATg TAC AT |
| Size: | 11.4 kb |
| Exon 11 | |

BClEX11

| Exon | Frag Primers 5′→3′ | | size | Tm (% UF) |
|---|---|---|---|---|
| 11.1 | | | | |
| [GC 3] | ACCTTGTTATTTTTGTATATTT | 22 | 347 | 40.99 |
| [GC 13] | TTGCTAAGCCAGGCTGTT | 18 | | |
| 11.2 | | | | |
| [GC 3] | ATACTCATGCCAGCTCATTA | 20 | 461 | 40.74 |
| [GC 12] | AACGTCCAATACATCAGCTA | 20 | | |
| 11.3 | | | | |
| | CATGCTCAGAGAATCCTAGA | 20 | 438 | 35.04 |
| [GC 3] | CTGTGGCTCAGTAACAAATG | 20 | | |
| 11.4 | | | | |
| [GC 12] | TCACTCCAAATCAGTAGAGA | 20 | 476 | 34.85 |
| [GC 3] | TACTGCTGCTTATAGGTTCA | 20 | | |
| 11.5 | | | | |
| [GC 3] | GAAAGCAGATTTGGCAGTTC | 20 | 468 | 33.66 |
| [GC 11] | CTGACTGGCATTTGGTTGTA | 20 | | |
| 11.6 | | | | |
| [GC 3] | GAATAGGCTGAGGAGGAAGT | 20 | 410 | 40.51 |
| [GC 13] | CTCTTGGAAGGCTAGGATTG | 20 | | |
| 11.7 | | | | |
| [GC 3] | ACAGCGATACTTTCCCAGAG | 20 | 345 | 36.45 |
| | TGCCTTCCCTAGAGTGCTAA | 20 | | |
| 11.8 | | | | |
| | TTGCAAACTGAAAGATCTGT | 20 | 365 | 38.37 |
| [GC 3] | GCTTTGAAACCTTGAATGTA | 20 | | |
| 11.9 | | | | |
| [GC 13] | GTCGGGAAACAAGCATAGAA | 20 | 422 | 40.40 |
| [GC 4] | TTGCCTCTGAACTGAGATGA | 20 | | |
| 11.10 | | | | |
| [GC 12] | TAATATCACTGCAGGCTTTC | 20 | 292 | 35.93 |
| [GC 1] | TTCCTCAAAGTTTTCCTCTA | 20 | | |
| 11.11 | | | | |
| [GC 1] | TCCCATCAAGTCATTTGTTA | 20 | 390 | 33.06 |
| | TTCCAGGAAGACTTTGTTTA | 20 | | |
| 11.12 | | | | |
| [GC 12] | TAATGAAGTGGGCTCCAGTA | 20 | 309 | 33.22 |
| [GC 1] | CTTCCCATAGGCTGTTCTAA | 20 | | |
| 11.13 | | | | |
| [GC 1] | GCAAGAATATGAAGAAGTAG | 20 | 305 | 37.43 |
| | CAAATGTGTATGGGTGAAAG | 20 | | |
| 11.14 | | | | |
| [GC 1] | AGACACCTGATGACCTGTTA | 20 | 378 | 43.03 |
| [GC 12] | TCTCCTCTGTGTTCTTAGAC | 20 | | |
| 11.15 | | | | |
| | CCTTTCACCCATACACATTT | 20 | 460 | 39.33 |
| [GC 8] | GACTGATGCCTCATTTGTTT | 20 | | |
| 11.16 | | | | |
| [GC 3] | CTCAGGAACATCACCTTAGT | 20 | 356 | 44.00 |
| [GC 16] | ATAAATAGACTGGGCCACAC | 20 | | |

All exons excluding exon 11

BRCAONE

| Exon | Frag Primers 5′→3′ | | size | Tm (% UF) |
|---|---|---|---|---|
| 2 1 | | | | |
| [GC 1] | TATATATGTTTTTCTAATGTGT | 22 | 203 | 34.64 |
| [GC 12] | TCCCAAATTAATACACTCTT | 20 | | |
| 3 1 | | | | |
| [GC 12] | GAGCCTCATTTATTTTCT | 18 | 269 | 37.22 |
| [GC 4] | ATTTTTCGTTCTCACTTA | 18 | | |
| 5 1 | | | | |
| [GC 4] | TATTTGCCTTTTGAGTAT | 18 | 305 | 26.69 |
| [GC 12] | TCTGATGAATGGTTTTAT | 18 | | |
| 6 1 | | | | |
| [GC 8] | ACTTGCTGAGTGTGTTTC | 18 | 213 | 35.52 |
| | GCACTTGAGTTGCATTCT | 18 | | |
| 7 1 | | | | |
| [GC 3] | TACATTTTTCTCTAACTGC | 19 | 250 | 32.67 |
| | GAAGAAAACAAATGGTTTT | 19 | | |
| 8 1 | | | | |
| | GGAGGAAAAGCACAGAAC | 18 | 248 | 40.51 |
| [GC 3] | CCAGCAATTATTATTAAATACTT | 23 | | |
| 9 1 | | | | |
| [GC 3] | CAGTAGATGCTCAGTAAA | 18 | 242 | 24.26 |
| | AATACCAGCTTCATAGAC | 18 | | |
| 10 1 | | | | |
| [GC 4] | CTGCATACATGTAACTAG | 18 | 229 | 38.30 |
| | CTACCCACTCTCTTTTCA | 18 | | |
| 12 1 | | | | |
| [GC 4] | AGTTGCAGCGTTTATAGT | 18 | 289 | 48.54 |
| [GC 13] | CAGCAAACCTAAGAATGT | 18 | | |
| 13 1 | | | | |
| [GC 4] | GCTTCTCAAAGTATTTCA | 18 | 293 | 45.18 |
| | AGTGTTTGGCCAACAATA | 18 | | |
| 14 1 | | | | |
| [GC 4] | CCAATTTGTGTATCATAG | 18 | 417 | 30.78 |
| [GC 13] | AGTGTATAAATGCCTGTA | 18 | | |
| 15 1 | | | | |
| [GC 1] | TGGTTTTCTCCTTCCATTTA | 20 | 303 | 46.07 |
| [GC 16] | TGTTCCAATACAGCAGATGA | 20 | | |
| 16 1 | | | | |
| [GC 13] | CGTTGTGTAAATTAAACTTC | 20 | 427 | 47.49 |
| [GC 1] | AGTCATTAGGGAGATACATA | 20 | | |
| 17 1 | | | | |
| [GC 4] | TGTGCTAGAGGTAACTCA | 18 | 242 | 32.51 |
| [GC 11] | CTCATGTGGTTTTATGCA | 18 | | |
| 18 1 | | | | |
| [GC 12] | TTTCAACTTCTAATCCTTT | 19 | 194 | 36.32 |
| [GC 4] | GGAGAAATAGTATTATACT | 19 | | |
| 19 1 | | | | |
| | GTTCTTCTGCTGTATGTA | 18 | 178 | 32.32 |
| [GC 4] | CTGAATGAATATCTCTGG | 18 | | |
| 20 1 | | | | |
| [GC 4] | CTCTTTCTCTTATCCTGAT | 19 | 219 | 46.40 |
| | TGGTGGGGTGAGATTTTT | 18 | | |
| 21 1 | | | | |
| [GC 8] | ATTCCCCTGTCCCTCTCT | 18 | 172 | 49.95 |
| | CTGGAACTCTGGGGTTCT | 18 | | |
| 2 1 | | | | |
| [GC 4] | TGATTTTACATCTAAATGTC | 20 | 209 | 47.71 |
| [GC 13] | AGGAGAGAATATTGTGTC | 18 | | |
| 8 1 | | | | |
| [GC 12] | TAGTCCTACTTTGACACT | 18 | 275 | 49.47 |
| [GC 4] | AAATATTTAAAATGTGCCAA | 20 | | |
| 4 1 | | | | |
| [GC 13] | AATCTCTGCTTGTGTTCTCT | 20 | 325 | 59.79 |
| [GC 18] | ATTTAGTAGCCAGGACAGTA | 20 | | |

What is claimed is:

1. A method of enabling BRCA1 and h MLHI gene testing for gene sequence variation and/or mutations, that comprises, preparing appropriate test kit primers and solvents suitable for amplifying by long distance PCR the entire gene-coding region of each of BRCA1 and MLHI genes, to be followed by multiplex short PCR using the long PCR products as templates; providing in such test kit appropriate buffer and gel and solvent materials for use in electrophoresis in orthogonal dimensions for producing spot patterns indicative of gene sequence variations and/or mutations.

2. The method of claim 1 wherein the test kit is provided with non-denaturing gel and buffer materials suitable to enable combined mixtures of the multiplex groups of BRCA1 and MLHI to be subjected to the electrophoresis simultaneously together.

3. The method of claims wherein, the kits provide the Primer Pairs A and B listed in the specification for Long Distance and Short PCR, respectively.

4. Test kits for enabling BRCA1 and h MLHI gene testing prepared by the method of claim 3.
